(19) United States
(12) Patent Application Publication
(10) Pub. No.: US 2024/0217931 A1
(43) Pub. Date: Jul. 4, 2024

(54) A SOLUTION OF ALKYLATED PIPERIDINAMINE- AND PIPERIDINAMINIUM-DERIVATES FOR THE PRODUCTION OF A PURIFIED SOLUTION OF TEMPO-DERIVATES FOR THE USE AS ELECTROLYTE IN REDOX-FLOW CELLS

(71) Applicant: BASF SE, Ludwigshafen am Rhein (DE)

(72) Inventors: Harald WINSEL, Ludwigshafen am Rhein (DE); Alexander Michael HAYDL, Ludwigshafen am Rhein (DE); Peter OECHSLE, Ludwigshafen am Rhein (DE); Joaquim Henrique TELES, Ludwigshafen am Rhein (DE); Juergen HERBEL, Ludwigshafen am Rhein (DE); Rainer KLOPSCH, Ludwigshafen am Rhein (DE)

(21) Appl. No.: 18/285,871

(22) PCT Filed: Mar. 28, 2022
(86) PCT No.: PCT/EP2022/058054

§ 371 Date: Oct. 6, 2023

(30) Foreign Application Priority Data
Apr. 7, 2021 (EP) 21167181.3

(51) Int. Cl. C07D 211/58; C07D 211/94; H01M 8/08; H01M 8/18

(52) U.S. Cl. CPC C07D 211/58; C07D 211/94; H01M 8/08; H01M 8/188; H01M 2300/0002

(57) ABSTRACT

The present invention relates to a solution comprising water, N,N,N, 2,2,6, 6-heptamethyl-4-piperidinaminium chloride of formula (II) and low amounts of N,N,N, 2,2,6,6-hexamethyl-4-piperidinamine of formula (I), byproducts and, N,N,1,2,2,6,6,octamethyl-4-piperidinaminium chloride of formula (III), a process for the production of this solution and the use of this solution for the production of an aqueous mixture comprising inter alia the corresponding TEMPO-derivates of formula (IV), (V) and (VI) wherein this aqueous mixture can be used as electrolyte in one chamber of a redox-flow cell for storing electrical energy.

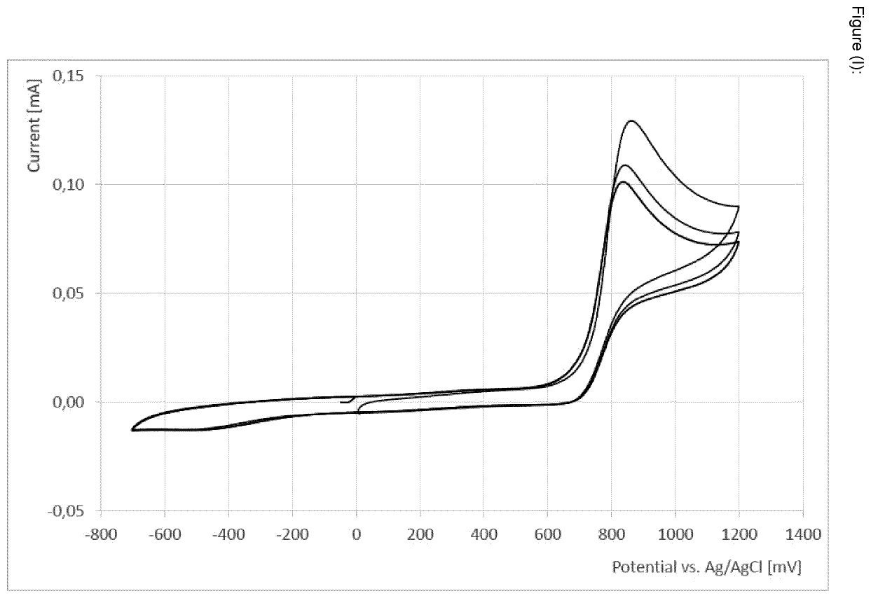

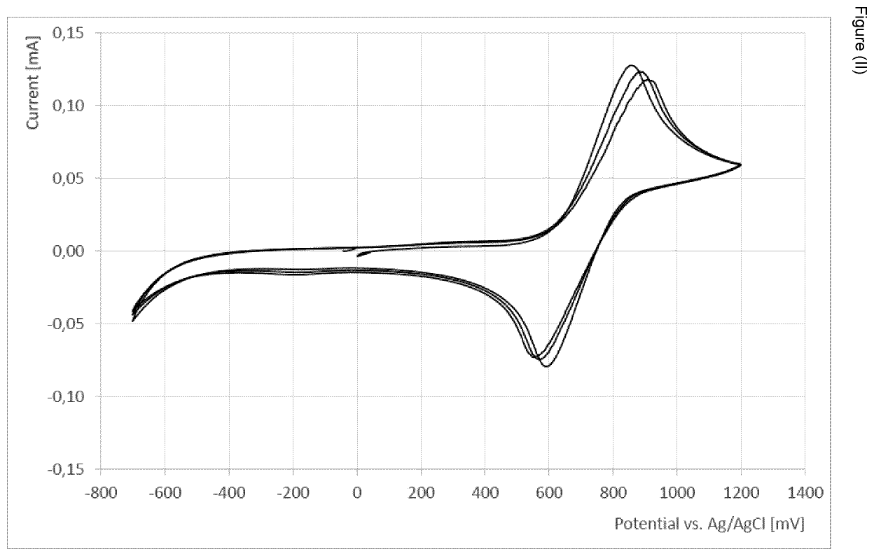

The present invention relates to a solution comprising water, N,N,N,2,2,6,6-heptamethyl-4-piperidinaminium chloride of formula (II) and low amounts of N,N,N,2,2,6,6-hexamethyl-4-piperidinamine of formula (I), byproducts and N,N,N,1,2,2,6,6-octamethyl-4-piperidinaminium chloride of formula (III), a process for the production of this solution and the use of this solution for the production of an aqueous electrolyte mixture comprising inter alia the corresponding TEMPO-derivates of formula (IV), (V) and (VI) wherein this aqueous mixture can be used as electrolyte in one chamber of a redox-flow cell for storing electrical energy.

Storing electrical energy for different kind of applications is a big problem that must be solved in the near future. WO 2014/26728 describes the use of new organic compounds as redox couple comprising 2,2,6,6-tetramethylpipderidinyl-oxyl (TEMPO)-derivates in redox-flow cells with a redox active potential separated from each other by using a membrane which selects the molecules by size as an easy and inexpensive way to provide a long-living redox-flow cell which will not have a negative impact on the environment.

WO 2018/028830 and T. Janoschka, M. D. Hager, U. S. Schubert describe in Angew. Chem. Int. ED 2016, 55, 14427-14430 a process of the production of 4-ammonium-2,2,6,6-tetraalkylpiperidinyl salts as typical TEMPO-derivates for using as electrolyte in the cathode chamber of a redox-flow cell. WO 2018/028830 discloses 3 different ways of production for this TEMPO-derivates. All these production ways use organic aprotic solvents like alcohols, ethers, nitriles, halogenated hydrocarbons, aromatic hydrocarbons, aliphatic hydrocarbons or mixtures of them. Furthermore, during the production some intermediate compounds are solids which have to be separated from the solvent before using them in the next reaction step. In the end a very pure final compound is obtained but during the production a lot of different solvents were used, and anions have to be exchanged. As the final 4-ammonium-2,2,6,6-tetraalkylpiperidinyl salt is used in a solution as an electrolyte in a redox-flow cell it is preferred to use it as an aqueous solution as water cannot be easily oxidized or reduced in the redox-flow cell, it is not flammable and therefore safer to handle, it is not toxic and very inexpensive and easily available.

WO 2021/197877 and WO2021/197876 describe ways how such aqueous solutions of TEMPO-derivates can be produced. These production ways result to aqueous solutions comprising the main TEMPO-derivate 2,2,6,6-tetramethylammonio-1-piperidinyloxy of formula (IV) as well as other TEMPO-derivates of formula (V) and (VI) in amounts of up to 20 wt.-% according to the total weight amounts of the aqueous solution. EP-A 20167458.7 and EP-A 20167462.9 describes that the TEMPO-derivates of formula (V) and (VI) which are the oxidized products of compound of formula (III) and (I) and which are not the redox active compounds in the chamber of the redox-flow cell will not influence the chemical redox potential of the TEMPO-derivate of formula (IV) and the cell. It was now found that TEMPO-derivates of formula (V) and (VI) will decomposed after oxidation and that the resulting decomposition products have an influence of the long-term stability of the redox-flow cell. Furthermore, it was found that the pure TEMPO-derivate (VI) show high decomposition energy of more than 1500 J/g, a 50% solution still more than 900 J/g together with a low onset temperature of 114° C. This means that mixture with a high amount of compound of formula (VI) may be potential explosive and not very stable. High efforts regarding safety has to be taken into account working with such high potential mixtures.

Therefore, it is preferred to have an aqueous mixture with a reduced amount of TEMPO-derivates of formula (V) and (VI). The separation of TEMPO-derivates of formula (V) and (VI) starting from the electrolyte mixture comprising water and all three TEMPO-derivates of formula (IV), (V) and (VI) is not possible via distillation as compounds of formula (IV), (V) and (VI) are salts which are not volatile. Purification can only be achieved via crystallization, as described in the state of the art. However, crystallization is a complex and expensive step, involving solids handling, solvent handling and recycling, and washing steps which inevitably lead to product losses and high solvent consumption. As mentioned previously the complexity will prevent to use this method for an industrial scale process.

Another way to obtain an aqueous mixture with reduced amounts of TEMPO-derivates of formula (V) and (VI) is to use mixtures which have very reduced amounts of compound of formula (I) and (III) in the final oxidation step. However, reducing the amount of both compounds of formula (I) and (III) just by adjusting the reaction conditions in the methylating step proved impossible. Increasing the amount of the methylating agent minimizes the amount of unconverted compound of formula (I) but leads to an unacceptable concentration of compound of formula (III). But separating compound of formula (III) from compound of formula (II) would only be possible by crystallization with all the additional complexity attached to it. This is thus not a viable technical option.

Decreasing the amount of methylating agent minimizes the amount of compound of formula (III) but leads to unacceptable concentrations of compound of formula (I). From the results obtained in the purification step of the production of compound of formula (I) by distillation it is known that compound of formula (I) forms an azeotrope with water, which contains only 1 wt.-% of compound of formula (I). It was thus expected, that to remove compound of formula (I) from the starting mixture one would have to evaporate 99 kg of water to remove 1 kg of unconverted compound of formula (I). The removal of compound of formula (I) by evaporation would thus be very costly in terms of energy.

Therefore, it is an object of the present invention to provide
an aqueous solution of compound of formula (II) with reduced amounts of compounds of formula (I) and (III) usable for the production of the aqueous mixture comprising the compound of formula (IV) and reduced amounts of compound of formula (V) and (VI) which can be used as electrolyte in a redox-flow cell and
a process for the production of this aqueous solution with the desired low levels of compounds of formula (I) and (III), and do this with lower complexity than required by crystallization and associated solids handling and with lower energy consumption than would be required by removing unconverted compound of formula (I) in the form of its azeotrope with water after the methylation step.

This problem will be solved by a solution comprising the following components:
a) water,
b) N,N,N,2,2,6,6,-hexamethyl-4-piperidinamine of formula (I)

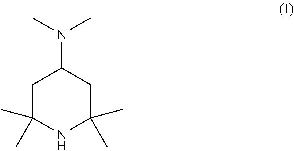

c) optionally byproducts which are not compound of formula (I), compound of formula (II) or compound of formula (III),
d) 90 to 98.5 wt.-% according to the sum of the total weight amounts of components b) to e) of N,N,N,2,2,6,6-heptamethyl-4-piperidinaminium chloride of formula (II)

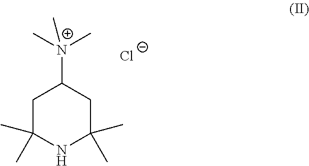

e) N,N,N,1,2,2,6,6,-octamethyl-4-piperidinaminium chloride of formula (III)

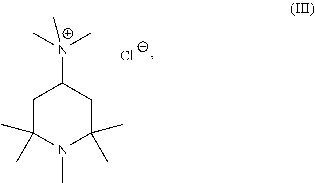

wherein the water content is in the range from 40 to 70 wt.-% according to the solution and the mass ratio between component of the formula (I) to compound of formula (III) is smaller than 1 and the mass ration between compound of formula (III) to compound of formula (II) is smaller than 0.04.

The inventive solution will be advantageous if the content of compound of formula (II) is in the range of 95 to 98.5 wt.-% according to the sum of the total weight amounts of components b) to e).

The inventive solution will be advantageous, if the content of water is in the range of 40 to 60 wt.-% according to the solution.

A further embodiment of the invention is a process of the production of the inventive solution comprising the following steps
i) Introducing a starting mixture comprising
   α) 40 to 90 wt.-% according to the total weight amount of the starting mixture of water,
   β) compound of formula (I),
   γ) optionally byproducts which are not compound of formula (I), compound of formula (II) and compound of formula (III),
   δ) compound of formula (II) and
   ϵ) compound of formula (III) wherein the mass ratio between compound of formula (I) to compound of formula (III) is greater than 1 and the mass ratio between compound of formula (III) to compound of formula (II) is smaller than 0.04, into a reactor vessel,
ii) removing a distillating mixture comprising water and parts of compound of formula (I) from the starting mixture by evaporation until the mass ratio of compound of formula (I) to compound of formula (III) in the resulting mixture at the bottom of the reactor vessel is smaller than 1,
iii) optionally adding water before, during and/or after the evaporation in step ii) in order to maintain the water content of the resulting mixture at the bottom of the reactor vessel between 40 to 70 wt.-% according to the total weight amount of the resulting mixture at the bottom of the reactor vessel.

The inventive process will be advantageous if no water is added before and/or during the step ii).

The inventive process will be advantageous if the starting mixture of step i) is obtained by methylation a reactant mixture comprising compound of formula (I) and optionally byproducts which are not compound of formula (I), compound of formula (II) or compound of formula (III) with a methylating agents in water, wherein the molar ration between compound of formula (I) and methylation agent is in the range from 1:0.90 to 1:1.

The inventive process will be advantageous if the methylating agent for the methylation in water to receive the starting mixture is selected from the group of methyl chloride and dimethyl sulfate. The inventive process will be advantageous if the methylation and the evaporation of the starting mixture are made in the same reaction vessel.

The inventive process will be advantageous if during the evaporation in step ii) a packed column or an evaporator is used.

The inventive process will be advantageous if the sump temperature during the evaporation in step ii) is in the range of 40 to 110° C. and the pressure during evaporation is in the range of 30 to 1000 mbar.

The inventive process will be advantageous if the water content of the resulting mixture after the evaporation in step ii) is in the range of 40 to 60 wt.-% according to the total weight amount of the resulting mixture after step ii).

A further embodiment of the invention is the use of the inventive solution for the production of an electrolyte mixture comprising
A) water,
B) 20 to 55 wt.-% according to the total weight amount of the electrolyte mixture of compound 2,2,6,6-tetramethylammonio)-1-piperidinyloxy of formula (IV)

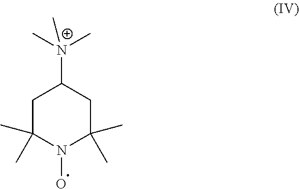

C) 0.1 to 6 wt.-% according to the total weight amount of the electrolyte mixture of an alkali metal cation
D) 0.5 to 12.5 wt.-% according to the total weight amount of the electrolyte mixture of compound N,N,N,1,2,2,6,6-octamethyl-4-piperidinammonium-1-oxide of formula (V)

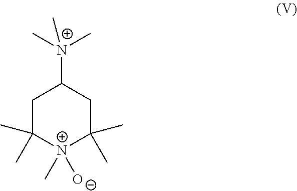

E) 0.1 to 20 wt.-% according to the total weight amount of the electrolyte mixture of compound 2,2,6,6-hexamethyl-4-(dimethylamino)-1-piperdinyloxy-N-oxide of formula (VI)

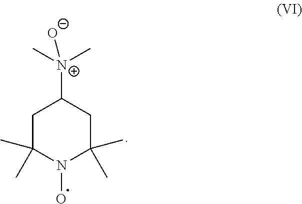

The inventive use will be advantageous if the amount of compound N,N,N,1,2,2,6,6-octamethyl-4-piperidinammonium-1-oxide of formula (V) in the electrolyte mixture is in the range of 0.5 to 6 wt.-% according to the total weight amount of the electrolyte mixture and the amount of compound 2,2,6,6-hexamethyl-4-(dimethylamino)-1-piperdinyloxy-N-oxide of formula (VI) in the electrolyte mixture is in the range of 0.1 to 0.5 wt.-% according to the total weight amount of the electrolyte mixture.

The inventive solution comprises water, N,N,N,2,2,6,6-hexamethyl-4-piperidinamine of formula (I), optionally byproducts which are not compound of formula (I), compound of formula (II) or compound of formula (III), 90 to 98.5 wt.-% according to the sum of the total weight amounts of components b) to e) of N,N,N,2,2,6,6-heptamethyl-4-piperidinaminium chloride of formula (II) and N,N,N,1,2,2,6,6-octamethyl-4-piperidinaminium chloride of formula (III). Preferably, the water content of the inventive solution is in the range from 40 to 70 wt.-%, particular in the range from 40 to 60 wt.-%, more particular in the range from 40 to 50 wt.-% according to the total weight amount of the solution.

The compound of formula (I) is N,N,2,2,6,6-hexamethyl-4-piperidinamine. It is one of the starting molecules to produce the inventive solution and will remain in small amounts in the inventive solution. Preferably the mass ratio between compound of formula (I) to compound of formula (III) in the inventive solution is smaller than 1, particular smaller than 0.7, more particular smaller than or equal to 0.5.

The inventive solution optionally comprises some byproducts which are not compound of formula (I), compound of formula (II) and compound of formula (III). These byproducts are obtained by the production of compound of formula (I) by using triacetonamine with dimethylamine according to known processes. Afterwards the compound of formula (I) is purified by distillation until a purity of at least 95%, preferred a purity of at least 97% is received. This distillation is very important as otherwise the amount of byproducts in the following steps will be too high. All products that are formed during this production process of compound of formula (I) and which are not compound of formula (I), compound of formula (II) and compound of formula (III) shall be understood as “byproducts” in this invention. These byproducts are for example selected from the group of triacetondiamine, triacetonamine, 4-hydroxy-2,2,6,6-tetramethylpiperidine, 2,2,6,6-tetramethylpiperidine, diisobutylketone, 2,6-dimethylhepta-2,5-dien-4-on, 4-amino-2,2,6,6-tetramethylpiperidine, 4-isopropyl-2,2,6,6-tetramethyl-1,3-dihydropyridine, 4-isopropyl-2,2,6,6-tetramethyl-1,3-dihydropiperidine and 4-(Methylamino)-2,2,6,6-tetramethylpiperidine. Preferably, the amount of byproducts in the inventive solution is in the range from 0 to 0.3 wt. %, particularly in the range from 0 to 0.2 wt.-% more particularly in the range from 0 to 0.15 wt. % according to the sum of the total weight amounts of compound of formula (I), compound of formula (II), compound of formula (III) and the byproducts.

The main component of the inventive solution is compound of formula (II) which is N,N,N,2,2,6,6-heptamethyl-4-piperidinaminium chloride. Preferably, the amount of compound of formula (II) is in the range of 90 to 98.5 wt.-%, particular in the range from 95 to 98.5 wt.-%, more particular in the range from 96 to 98.5 wt.-% according to the sum of the total weight amounts of compound of formula (I), compound of formula (II), compound of formula (III) and byproducts which are not compound of formula (I), compound of formula (II) or compound of formula (III) of the inventive solution.

The compound of formula (III) is N,N,N,1,2,2,6,6-octamethyl-4-piperidinaminium chloride. It is obtained as side product during the methylation of compound of formula (I) with a methylation agent. Preferably, the mass ratio of compound of formula (III) to compound of formula (II) is smaller than 0.04, particularly smaller than 0.03.

The sum of all amounts of compound of formula (I), (II), (III) and byproducts is preferably in the range from 40 to 60 wt.-%, particular in the range from 45 to 60 wt.-%, more preferably in the range from 50 to 60 wt. % according to total amount of the inventive solution.

The inventive solution is obtained by the inventive process. This inventive process comprises the following steps:
i) Introducing a starting mixture comprising
   α) 40 to 90 wt.-% according to the total weight amount of the starting mixture of water,
   β) compound of formula (I),
   γ) optionally byproducts which are not compound of formula (I), compound of formula (II) and compound of formula (III),
   δ) compound of formula (II) and
   ϵ) compound of formula (III) wherein the mass ratio between compound of formula (I) to compound of formula (III) is greater than 1 and the mass ratio between compound of formula (III) to compound of formula (II) is smaller than 0.04, into a reactor vessel,
ii) removing a distillating mixture comprising water and parts of compound of formula (I) from the starting mixture by evaporation until the mass ratio of compound of formula (I) to compound of formula (III) in the resulting mixture at the bottom of the reactor vessel is smaller than 1,
iii) optionally adding water before, during and/or after the evaporation in step ii) in order to maintain the water content of the resulting mixture at the bottom of the reactor vessel between 40 to 70 wt.-% according to the total weight amount of the resulting mixture at the bottom of the reactor vessel.

For the first step i) a starting mixture is used. “Starting mixture” in the inventive process shall mean a mixture comprising 40 to 90 wt.-% according to the total amounts of the starting mixture of water, compound of formula (I), compound of formula (II), compound of formula (III) and optionally byproducts wherein the mass ratio between compound of formula (I) to compound of formula (III) is higher than 1 and the mass ratio between compound of (III) to compound of formula (II) is smaller than 0.04.

The starting mixture is obtained by methylating a mixture comprising the compound of formula (I) an optionally byproducts with a methylation agent in water.

For the methylation of compound of formula (I) and optionally byproducts in water the methylation agent is selected from the group of methyl chloride, and dimethyl sulfate. The preferred methylation agent in water is methyl chloride.

For the methylation in water compound of formula (I) and optionally byproducts are dissolved in water and methylated with the methylation agent. Preferably, the aqueous solution of compound of formula (I) and optionally byproducts is methylated with 0.9 to 1.0 mol, particularly 0.95 to 1.0 mol, more preferably with 0.98 to 1.0 mol methylation agent per mol of compound of formula (I) in the methylation reaction. The phrase “compound of formula (I) in the methylation reaction” shall mean the amount of compound of formula (I) that is dissolved in water at the beginning of the methylation reaction and not the amount of compound of formula (I) that still remains in the solution after methylation. During the methylation in water the temperature of the reaction is preferably in the range of 0 to 60° C., particularly in the range from 0 to 40° C., more preferably between 15 to 25° C. The temperature can be controlled by external heating, cooling or by slowly adding the methylation agent to the aqueous mixture of compound of formula (I) so that the temperature will not rise above 60° C. Preferred is the slow addition of the methylation agent and the use of external cooling so that the temperature does not rise above 60° C. The mixture, which is obtained after methylation is completed, is the starting mixture, if the water content of this mixture is in the range of 40 to 90 wt.-% according to the total amount of the mixture.

In step ii) of the inventive process the starting mixture of step i) is heated up in order to remove a distillating mixture from the starting mixture by evaporation until the mass ratio of compound of formula (I) to compound of formula (III) in the resulting mixture at the bottom of the reactor vessel is smaller than 1. The “distillating mixture” shall mean a mixture that comprises water, parts of the compound of formula (I) and byproducts. Preferably the distillating mixture comprises water, parts of the compound of formula (I), byproducts and no parts of compound of formula (III), particular the distillating mixture consists of water, parts of compound of formula (I) and byproducts, more particular the distillating mixture consists of water and parts of compound of formula (I). Preferably, the amount of compound of formula (I) in the distillating mixture is in the range from 90 to 99.9 wt.-%, particular in the range from 91 to 98 wt.-%, more particular in the range from 92 to 97 wt.-% according to the total amount of compound of formula (I) in the starting mixture.

The content of compound of formula (I) and (III) in the resulting mixture is measured in step ii) of the inventive process by taking samples from the sump of the bottom of the reactor vessel. The “resulting mixture” shall mean every mixture comprising water, compound of formula (I), compound of formula (II), compound of formula (III) and optionally byproducts wherein the content of compound of formula (I) is smaller than in the starting mixture. Preferably, the reactor vessel has at least one side exit for taking samples during the evaporation of step ii) of the inventive process. Preferably, the samples will be taken and measured continuously during step ii) of the inventive process. Step ii) is finished when the mass ratio between compound of formula (I) to compound of formula (III) in the resulting mixture of the bottom of the reactor vessel is smaller than 1, preferably smaller than 0.7, particular smaller than 0.5.

Preferably, the water content of the resulting mixture after the evaporation of the distillating mixture is completed is in the range from 40 to 70 wt.-%, particular in the range of 40 to 60 wt.-% according to the total amount of the resulting mixture. In this special case the resulting mixture is the inventive solution.

Preferably, the temperature in the sump during the evaporation in step ii) is in the range from 40 to 110° C., particular in the range from 60 to 110° C. and more particular in the range from 90 to 110° C. Preferably, the pressure during the evaporation in step ii) is in the range from 30 to 1000 mbar, particular in the range from 700 to 1000 mbar, more particular in the range from 900 to 1000 mbar.

Preferably, a packed column or an evaporator is used for evaporating the distillating mixture off the starting mixture in step ii) of the inventive process.

In a special embodiment of the inventive process water can be added before, during and after the step ii) of the inventive process. The amount of added water is only the amount that is missing in the resulting mixture to have a water content of 40 to 70 wt.-%, preferably 40 to 60 wt.-% according to the total amount of the resulting mixture. If water is added during the evaporation of step ii) of the inventive process the reactor vessel will have a further side exit in order to add the water continuously to the resulting mixture into the reaction vessel. The water that is added in step iii) of the inventive process can be added as liquid water and/or as vapor. Preferably, liquid water is used in step iii) of the inventive process. Preferably, no water is added before and/or during the step ii) of the inventive process, particular water is added to the resulting mixture after the evaporation of step ii) of the inventive process, more particular the amount of water that is added to the resulting mixture after the evaporation of step ii) of the inventive process is as high as the amount of the distillating mixture evaporated off the starting mixture in step ii) of the inventive process.

Preferably, the temperature in the sump during the evaporation in step ii) is in the range from 40 to 110° C., particularly in the range from 60 to 110° C. and more particularly in the range from 90 to 110° C. Preferably, the pressure during the evaporation in step ii) is in the range from 30 to 1000 mbar, particularly in the range from 700 to 1000 mbar, more particularly in the range from 900 to 1000 mbar.

The methylation of compound of formula (I) and optionally byproducts with a methylation agent in water and the separation of the distilling mixture in step ii) of the inventive process can be made in different or the same reactor vessel. Preferable, the methylation of compound of formula (I) and optionally byproducts with a methylation agent in water are made in the same reactor vessel, more particular the methylation step and the separation of the distilling mixture in step ii) of the inventive process are made in only one—the same—reactor vessel.

In further embodiment of the invention the inventive solution is used for the production of an electrolyte mixture comprising water, 20 to 55 wt.-% according to the total weight amount of the electrolyte mixture of compound 2,2,6,6,-tetramethylammonio)-1-piperidinyloxy of formula (IV), 0.1 to 6 wt.-% according to the total weight amount of the electrolyte mixture of an alkali metal cation, 0.5 to 12.5 wt.-% according to the total weight amount of the electrolyte mixture of compound N,N,N,1,2,2,6,6-octamethyl-4-piperidinammonium-1-oxide of formula (V) and 0.1 to 20 wt.-% according to the total weight amount of the electrolyte mixture of compound 2,2,6,6-hexamethyl-4-(dimethylamino)-1-piperdinyloxy-N-oxide of formula (VI). The “electrolyte mixture” shall mean every mixture that comprises water, compound of formula (IV), (V), (VI) and the alkali metal cation wherein the amounts of compound of formula (IV), (V), (VI) and the alkali metal cation are in the above mentioned ranges. Preferably, the amount of compound N,N,N,1,2,2,6,6-octamethyl-4-piperidinammonium-1-oxide of formula (V) in the electrolyte mixture is in the range of 0.5 to 6 wt.-% according to the total weight amount of the electrolyte mixture and the amount of compound 2,2,6,6-hexamethyl-4-(dimethylamino)-1-piperdinyloxy-N-oxide of formula (VI) in the electrolyte mixture is in the range of 0.1 to 0.5 wt.-% according to the total weight amount of the electrolyte mixture.

With the inventive solution the electrolyte mixture can be produced. The electrolyte mixture can be used as an electrolyte in a redox-flow cell. Preferably the electrolyte mixture is used as catholyte in such a redox-flow cell. The redox-flow cell is normally built up by using two chambers for catholyte and anolyte solution each connected via a pump to a storage tank for catholyte and anolyte solution respectively. Both chambers are separated by an ion-conducting membrane and equipped with electrodes. In the cathode chamber and the connected storage tank of the cathode the electrolyte mixture is filled. In the anode chamber and the connected storage tank of the anode the electrolyte for the anode is filled. The redox active compounds in the redox-flow cell change during charging and discharging between their different redox levels. For discharging the electrolyte has to be pumped from the storage tank to the electrode while for charging the inverse process is used. Therefore, the redox-flow cell comprising the electrolyte mixture as electrolyte, which is received by using the inventive solution, is an easy and multifunctional way to storage electrical energy for different applications.

### EXAMPLES

### Generals

### ¹H-NMR Method:

### ¹H-NMR Data of Compound of Formula (II):

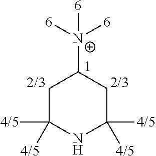

¹H-NMR (500 MHz, D₂O): δ [ppm]=3.68 (tt, J=12.5 Hz, 2.8 Hz 1H, H₁), 3.07 (s, 9H, H₆), 2.02-2.08 (m, 2H, H₃), 1.32 (t, J=12.5 Hz, 2H, H₂), 1.14 (s, 6H, H₅), 1.12 (s, 6H, H₄).

### ¹H-NMR Data of Compound of Formula (III):

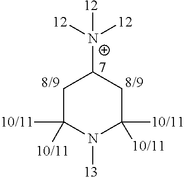

¹H-NMR (500 MHz, D₂O): δ [ppm]=3.62 (tt, J=12.5 Hz, 3.1 Hz, 1H, H₇), 3.00 (s, 9H, H₁₂), 2.15 (s, 3H, H₁₃), 2.08-2.02 (m, 2H, H₈), 1.55 (t, J=12.5 Hz, 2H, H₈), 1.15 (s, 6H, H₁₁), 1.05 (s, 6H, H₁₀).

### ¹H-NMR Data of Compound of Formula (I):

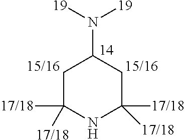

¹H-NMR (500 MHz, D₂O): δ [ppm]=2.83 (tt, J=12.3 Hz, 3.2 Hz, 1H, H₁₄), 2.27 (s, 6H, H₁₉), 1.88 (dd, J=12.7 Hz, 3.2 Hz, 2H, H₁₅), 1.28 (s, 6H, H₁₇), 1.24 (s, 6H, H₁₈), 1.17 (dd, J=12.7 Hz, 12.3 Hz, 6H, H₁₆).

The molar ratio of compound of formula (I), (II) and (III) can be determined most conveniently by comparing the integrals of the ¹H-NMR signals at δ=3.68 ppm (1H from compound of formula (II)), 2.15 ppm (3H from compound of formula (III)) and 2.27 ppm (6H from compound of formula (I)).

Thus the molar ratio of compound of formula (I):(II):(III) is the same as the ratio of the following integrals:

(integral of signal at δ=3.68 ppm from compound of formula (II)):(integral of signal at δ=2.15 ppm from compound of formula (III))/3:(integral of signal at δ=2.27 ppm from compound of formula (I))/6

### Cyclic Voltammetry Method:

The solution obtained from the respective example is diluted with 0.1 mol/L aqueous sodium chloride solution until the concentration of the N-oxyl compounds is 1.0 wt.-%. Said solution is placed in an electrochemical cell equipped with a standard 3 electrode setup (working electrode: glassy carbon (ø=2 mm), counter electrode: platinum wire, reference electrode: Ag/AgCl, 3 mol/L KCl in water). The potential is ramped to 1200 mV and then cycled between 1200 mV and −700 mV at a scan rate of ±20 mV/s (in total 3 cycles) using PGU 20V-2A-E potentiostat (IPS).

### Method for Gas-Chromatographic Analysis

The GC-analyzes were carried out using the following method: column Restek Rtx 5 Amine, column length 30 m, column diameter 0.32 mm, film thickness 1.5 μm; temperature program: start at 60° C., ramp with 5 K/min to 190° C., ramp with 10 K/min to 280° C., total runtime 40 min.

### Example 1 (Comparative)

In this example a crude product mixture obtained in the synthesis of compound of formula (I) by reductive amination of triacetone amine with dimethylamine was used. This crude product mixture containing 82 wt.-% of compound of formula (I) and 10 wt.-% water, the remaining being other organic impurities was loaded into a batch distillation apparatus with a high efficiency rectification tower (ca. 110 theoretical stages) and distilled at ambient pressure. The first fractions obtained consisted mainly of water which contained 0.8 to 1.0 wt.-% of compound of formula (I). An increase of the reflux ratio did not change the content of compound of formula (I) in the distillate. Separate experiments confirmed that at ambient pressure compound of formula (I) forms an azeotrope with water, which contains ca. 1 wt.-% of compound of formula (I). Therefore, it was expected that in order to remove compound of formula (I) from an aqueous mixture containing compound of formula (I) it would be necessary to evaporate 99 kg of water per kg of (I) to be removed.

### Example 2 (Comparative)

A starting mixture comprising compound of formula (II) was obtained by methylating an aqueous solution of compound of formula (I) (purity: 99.7%) with 0.9 equivalents of methyl chloride as described in EP-A 20167458.7. The product obtained contains 49.3 wt.-% of water as determined by Karl-Fischer titration. The organic material contains (water excluded and as determined by ¹H-NMR: 500 MHz in D₂O) 92.2% of compound of formula (II), 5.0% of compound of formula (I), 2.5% of compound of formula (III). The sum of all other byproducts was less than 0.3%.

100 g of the above mixture is charged to a batch distillation apparatus with a column having only 10 theoretical stages and diluted with 500 g of water. Distillation at ambient pressure was started and five fractions, each of them containing approximately 100 g were collected. The sump temperature remained at approximately 100° C. After the collection of each fraction a sample of the sump was taken and analyzed by NMR (¹H-NMR: 500 MHz in D₂O) to determine the contents of compounds of formula (I), (II) and (III) in the sump. Table 1 shows the composition of the sump after each distillation faction 1 to 5 was removed

From the data of table 1 the amount of water which had to be distilled off in order to remove compound of formula (I) from the sump product is 173 g water per gram of compound of formula (I) removed. Considering the lower number of theoretical stages, the amount of water which has to be distilled off to remove compound of formula (I) is in the range expected based on the results of example 1.

TABLE 1

| | | | | Percentage of |
|---|---|---|---|---|
| | Composition of the sump (water free basis) | | | compound of |
| | Compound of | Compound of | Compound of | formula (I) |
| | formula (I) | formula (II) | formula (III) | removed |
| | wt.-% | wt.-% | wt.-% | % |
| Initial composition ^{a)} | 5.0 | 92.2 | 2.5 | 0 |
| After distilling 1^{st} fraction | 3.1 | 94.0 | 2.8 | 34 |
| After distilling 2^{nd} fraction | 1.9 | 95.24 | 2.9 | 60 |
| After distilling 3^{rd} fraction | 1.1 | 96.00 | 2.9 | 77 |
| After distilling 4^{th} fraction | 0.5 | 96.62 | 2.9 | 90 |
| After distilling 5^{th} fraction | 0.0 | 97.09 | 2.9 | 100 |

^{a)} Besides compound of formula (I), (II) and (III) the mixture contained in sum 0.3 wt.-% of other impurities.

### Example 3 (Inventive)

Example 2 was repeated, but now the distillation apparatus was loaded with 500 g of the same aqueous solution, but no water was added. Distillation under ambient pressure was then started. The sump temperature, which was initially at 100° C., steadily increased and the distillation was stopped when the sump temperature reached 107° C. (note that this is well below the boiling point of compound of formula (II): 213.5° C.). Until this point 94.1 g of distillate had been collected. Heating was stopped and 94.1 g of pure water were added to the sump. This is necessary to avoid precipitation of compound of formula (II) upon cooling.

The distillate was then analyzed via GC. Almost the entire amount of compound of formula (I) contained in the starting mixture and a large part of the minor by-products which are not salts, were contained in the distillate.

After cooling to ambient temperature, the sump was analyzed with NMR (¹H-NMR: 500 MHz in D₂O). It contained on a water free basis: 96.6% of compound of formula (II), 0.5% of compound of formula (I) and 2.8% of compound of formula (III). The sum of all other byproducts was less than 0.06%. The amount of water that had to be evaporated in this case was only 7.1 g water per gram of compound of formula (I) removed.

### Example 4 (Inventive)

184.3 g (0.976 mol) of compound of formula (I) with a purity of 97.6% (measured by GC) are dissolved in 315 ml of water. It contains the following impurities: 4-amino-2,2,6,6-tetramethyl-piperidine (0.09 wt.-%), 2,2,6,6-tetramethyl-4-piperidone (0.22 wt.-%), 4-Hydroxy-2,2,6,6-tetramethyl-piperidine (0.25 wt.-%), N,2,2,6,6-Pentamethyl-4-piperidinamine (1.25 wt.-%), 4-Isopropyl-2,2,6,6-tetramethyl-piperidine (0.345 wt.-%) besides a few small unidentified by-products. This aqueous mixture was stirred in a reactor vessel connected with a distillation column and 22.4 L (0.999 mol) of methyl chloride as a gas are added to this aqueous mixture during a time frame of 12 h. During this methylation reaction the temperature of the mixture is maintained at 20° C. using external cooling devices. Afterwards the reaction vessel is purged with nitrogen in order to remove remaining methyl chloride out of the reactor vessel. A sample of the product mixture was analyzed by NMR (¹H-NMR: 500 MHz in D₂O). On a water free basis, the mixture contains 81.9% of compound of formula (II), 15.7% of compound of formula (I) and 0.8% of compound of formula (III). The sum of all the other small byproducts was less than 1.5% of the total. The water content as measured by Karl-Fischer titration was 66.1 wt.-%.

This mixture remains in the reactor vessel with distillation column and is heated up under normal pressure. Distillate is gathered until the temperature of the sump in the reactor vessel reaches 107° C. Then distillation was stopped, and the distillate was analyzed: The resulting weight of the distillate was 203.9 g. The distillate was analyzed via GC. Approximately 97% of the amount of compound of formula (I) which was initially contained the starting mixture and a large part of the other by-products, were found in the distillate.

The material which remained in the sump weighed 333.5 g. A sample was taken and analyzed via NMR (¹H-NMR: 500 MHz in D₂O). On a water free basis, it contained: 98.1% of compound of formula (II), 0.5% of compound of formula (I) and, 1.3% of compound of formula (III). The sum of all other small by-products was less than 0.12%. The water content as determined by Karl-Fischer titration was 46.7 wt.-%. The distillation thus removed 97.3% of the compound of formula (I) contained in the reaction mixture. The amount of water which had to be evaporated amounted to 4.7 g water per g of (I) removed.

### Example 5

Cyclic voltammetry of compound of formula (V) is measured (see FIG. I). The cyclic voltammogram in FIG. I is irreversible which shows that the compound of formula (V) decomposes upon oxidation and this has a negative influence on the long-term stability of the redox-flow cell.

### Example 6

Cyclic voltammetry of compound of formula (VI) was also measured (see FIG. II).

1.-13. (canceled)

14. Solution comprising the following components:
a) water,
b) N,N,N,2,2,6,6-hexamethyl-4-piperidinamine of formula (I) 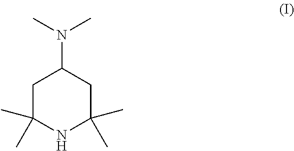
c) optionally byproducts which are not compound of formula (I), compound of formula (II) or compound of formula (III),
d) 90 to 98.5 wt.-% according to the sum of the total weight amounts of components b) to e) of N,N,N,2,2,6,6-heptamethyl-4-piperidinaminium chloride of formula (II) 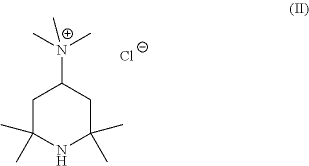
e) N,N,N,1,2,2,6,6,-octamethyl-4-piperidinaminium chloride of formula (III) 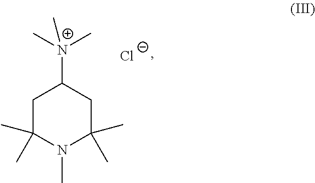
wherein the water content is in the range from 40 to 70 wt.-% according to the solution and the mass ratio between component of the formula (I) to compound of formula (III) is smaller than 1 and the mass ration between compound of formula (III) to compound of formula (II) is smaller than 0.04.

15. The solution of claim 14, wherein the content of compound of formula (II) is in the range of 95 to 98.5 wt.-% according to the sum of the total weight amounts of components b) to e).

16. The solution of claim 14, wherein the content of water is in the range of 40 to 60 wt.-% according to the solution.

17. Process of the production of the solution according to claim 14 comprising the following steps
i) Introducing a starting mixture comprising
α) 40 to 90 wt.-% according to the total weight amount of the starting mixture of water,
β) compound of formula (I),
γ) optionally byproducts which are not compound of formula (I), compound of formula (II) and compound of formula (III),
δ) compound of formula (II) and
ϵ) compound of formula (III)
wherein the mass ratio between compound of formula (I) to compound of formula (III) is greater than 1 and the mass ratio between compound of formula (III) to compound of formula (II) is smaller than 0.04, into a reactor vessel,
ii) removing a distillating mixture comprising water and parts of compound of formula (I) from the starting mixture by evaporation until the mass ratio of compound of formula (I) to compound of formula (III) in the resulting mixture at the bottom of the reactor vessel is smaller than 1,
iii) optionally adding water before, during and/or after the evaporation in step ii) in order to maintain the water content of the resulting mixture at the bottom of the reactor vessel between 40 to 70 wt.-% according to the total weight amount of the resulting mixture at the bottom of the reactor vessel.

18. The process according to claim 17, wherein no water is added before and/or during the step ii).

19. The process according to claim 17, wherein the starting mixture of step i) is obtained by methylation a reactant mixture comprising compound of formula (I) and optionally byproducts which are not compound of formula (I), compound of formula (TI) or compound of formula (III) with a methylating agents in water, wherein the molar ration between compound of formula (I) and methylation agent is in the range from 1:0.90 to 1:1.

20. The process according to claim 17, wherein the methylating agent for the methylation in water to receive the starting mixture is selected from the group of methyl chloride, and dimethyl sulfate.

21. The process according to claim 17, wherein the methylation and the evaporation of the starting mixture are made in the same reaction vessel.

22. The process according to claim 17, wherein during the evaporation in step ii) a packed column or an evaporator is used.

23. The process according to claim 17, wherein the sump temperature during the evaporation in step ii) is in the range of 40 to 110° C. and the pressure during evaporation is in the range of 30 to 1000 mbar.

24. The process according to claim 17, wherein the water content of the resulting mixture after the evaporation in step ii) is in the range of 40 to 60 wt.-% according to the total weight amount of the resulting mixture after step ii).

25. Use of the solution according to claim 14 for the production of a electrolyte mixture comprising
A) water,
B) 20 to 55 wt.-% according to the total weight amount of the electrolyte mixture of compound 2,2,6,6-tetramethylammonio)-1-piperidinyloxy of formula (IV) 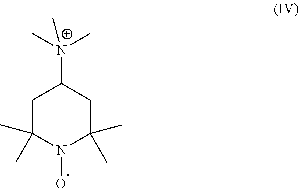
C) 0.1 to 6 wt.-% according to the total weight amount of the electrolyte mixture of an alkali metal cation
D) 0.5 to 12.5 wt.-% according to the total weight amount of the electrolyte mixture of compound N,N,N,1,2,2,6,6-octamethyl-4-piperidinammonium-1-oxide of formula (V) 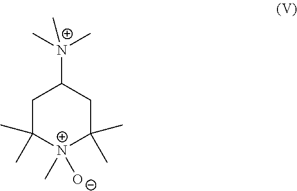
E) 0.1 to 20 wt.-% according to the total weight amount of the electrolyte mixture of compound 2,2,6,6-hexamethyl-4-(dimethylamino)-1-piperdinyloxy-N-oxide of formula (VI)

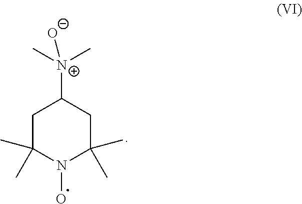

26. The use according to claim 25, wherein the amount of compound N,N,N,1,2,2,6,6-octa-methyl-4-piperidinammonium-1-oxide of formula (V) in the electrolyte mixture is in the range of 0.5 to 6 wt.-% according to the total weight amount of the electrolyte mixture and the amount of compound 2,2,6,6-hexamethyl-4-(dimethylamino)-1-piperdinyloxy-N-oxide of formula (VI) in the electrolyte mixture is in the range of 0.1 to 0.5 wt.-% according to the total weight amount of the electrolyte mixture.
